(19) United States
(12) Patent Application Publication
(10) Pub. No.: US 2003/0133901 A1
(43) Pub. Date: Jul. 17, 2003

(54) Composition of material for cleansing cream

(76) Inventors: Yeo Yong Yun, Los Angeles, CA (US); Eugene Oak, Los Angeles, CA (US)

Correspondence Address: Eugene Oak, Phd., J.D. Patent Attorney, 610 S. Van Ness Ave., Los Angeles, CA, 90005, US

(21) Appl. No.: 10/046,043

(22) Filed: Jan. 15, 2002

(51) Int. Cl. A61K 7/075; A61K 7/08; A61K 35/78

(52) U.S. Cl. 424/70.22; 424/765; 424/745

(57) ABSTRACT

A unique composition of material for manufacturing a cleansing cream has an excellent cleansing effect of greasy dirt, pigments and cosmetic residues from human skin. The basic composition of material of this invention to render the cleansing effect of greasy dirt, pigments and cosmetic residues from human skin is composed of ducks' fat oil and sodium carbonate, Na₂CO₃. The cleansing cream of this invention is excellent in cleansing greasy, viscous high molecular organic materials from not only human skin but also leathers, cloths, tiles, plastic and metal surfaces, by just pasting the cream and scrubbing. The cream can be wiped out with a tissue or cloth towel, rinsed with warm water. Alternatively, the cream can be allowed to stay on the skin and surface following the cleansing. The cleansing cream of this invention is non-toxic, non-allergic to human skin and environmentally affirmative. The cleansing cream of this invention is especially suitable for washing out of disguise pigments from a soldier's face.

### BACKGROUND OF THE INVENTION

1. Field of Invention

A composition of material for manufacturing cleansing cream having an excellent cleansing effect of greasy dirt, paints and cosmetic residues from human skin includes but not limited to a duck oil and sodium carbonate. The basic composition of material of this invention to render cleansing effect of 1 5 greasy dirt, pigments and cosmetic residues from human skin is composed of, including but not limited to, ducks' fat oil and sodium carbonate, Na₂CO₃.

2. Description of the Prior Art

U.S. Pat. No. 273,329 to Johnston illustrates the method of manufacturing toilet-soap, using tallow as one of the raw materials. It teaches to mix various oils such as lavender oil, petroleum oil, rosin, and citronella oil. No indication of utilizing duck's oil is taught in the patent.

U.S. Pat. No. 4,297,374 to Wess teaches a method of manufacturing skin moisturizing and cleansing cream by blending a quantity of fresh bananas or avocados with smaller amounts of baking powder, orange juice and solid or liquid vegetable shortening. No animal fat is used for the Wess' cleansing cream.

U.S. Pat. No. 4,765,922 to Contamin, et al. illustrates a skin cleansing composition in the form of a rod or stick comprising a lactate containing a fatty phase, a water phase and a consistency agent. Animal originated purcellin oil, perhydrosqualene, animal originated bees wax, spermaceti, and lanolin wax were mixed with various vegetable oil and mineral oils. Use of duck's oil was not suggested in Contamin.

U.S. Pat. Nos. 4,808,322 and 4,941,990 to McLaughlin illustrate a skin cleansing-cream conditioning bar consisting essentially of an anionic surfactant material and mixtures of monocarboxylate salt. None of naturally occurring components was used to form the non-foaming cleansing bar.

None of prior art reports utilization of duck's oil in conjunction with sodium carbonate for manufacturing a cleansing cream.

### SUMMARY OF THE INVENTION

Therefore, it is the object of this invention to provide a new composition of material for manufacture of cleansing cream having excellent cleansing power for greasy dirt, pigments and cosmetic residuals from human skin without contaminating the environment.

The basic composition of material of this invention to render cleansing effect of greasy dirt, pigments and cosmetic residues from human skin is composed of ducks' fat oil and sodium salt of organic and inorganic, including but not limited to, sodium carbonate, sodium sulfate and sodium hydroxide. Small amount of natural aroma is added for cosmetic application.

The cleansing cream of this invention is excellent in cleansing greasy viscous high molecular organic materials from human skin, leathers, cloths, tiles, plastic and metal surfaces by just pasting the cream and scrubbing.

The cream can be wiped out with a tissue or cloth towel, rinsed with warm water. Alternatively, the cream can be allowed to stay on the skin and surface following the cleansing.

The cleansing cream of this invention is non-toxic, and non-allergic to human skin. The cleansing cream of this invention easily washes out disguise pigments from a soldier's face and make-up colors from a woman's face, without itching and allergy by the residual.

### DETAILED DESCRIPTION OF THE PREFERRED EMBODIMENTS

A mass fat from inner body of duck's abdomen and subcutaneous organisms are collected by an extraction process. Collected fat is solubilized at 100˜120° C., followed by treatment with carbon black to eliminate the smell of duck.

Small amount of sodium salts, sodium carbonate, sodium sulfate, sodium hydroxide, is added to the hot oil. The oil becomes white and creamy as one of the sodium salt is added, None of artificial foaming agent is utilized to prevent environmental contamination.

Tar painted on a wood is easily erased through pasting of the oil and sodium carbonate mixture on the wood. Hands and face stained with oily paint are cleaned by just wash with the oil and sodium carbonate mixture.

Make-ups on human face are easily erased by use of the mixture. Small amount of vegetarian oil is mixed for the application of cosmetic cleansing cream.

Best mode of the composition of this invention is 0.5 wt % of Na₂CO₃ in duck's oil. Trace amount of water depresses the cleansing power.

What is claimed is:

1. A composition of material for manufacturing cleansing cream non-toxic and non-allergic to human skin, having excellent cleansing effect of greasy dirt, pigments and cosmetic residues on a human skin, consists of 99.5 wt % of duck's oil, 0.45 wt % of sodium salts of organic and inorganic and 0.05 wt % of vegetarian oils, which can be wiped out with a tissue and rinsed with warm water.

2. The content of sodium salts in claim 1 is in the range of 0.001 to 25 wt %.

3. The content of sodium salts in claim 1 is in the range of 0.1 to 10 wt %.

4. The sodium salts in claim 1 is sodium carbonate.

5. The sodium salts in claim 1 is sodium sulfate.

6. The sodium salts in claim 1 is sodium hydroxide.

7. The content of vegetarian oil in claim 1 is in the range of 0.001 to 30%.

8. The vegetarian oil in claim 1 is lavender oil.

9. The vegetarian oil in claim 1 is rose oil.
